(19) United States
(12) Patent Application Publication
(10) Pub. No.: US 2020/0229480 A1
(43) Pub. Date: Jul. 23, 2020

(54) NUTRITIONAL COMPOSITIONS WITH PARTIALLY HYDROLYSED PROTEINS FOR USE IN INDUCING GLUCOSE AND/OR INSULIN RESPONSE(S) CLOSE TO THE ONES OBSERVED WITH HUMAN MILK

(71) Applicant: SOCIETE DES PRODUITS NESTLE S.A., Vevey (CH)

(72) Inventors: Yassaman Shahkhalili Dulloo, La Tour de Peilz (CH); Catherine Mace, La Conversion (CH)

(21) Appl. No.: 16/486,247

(22) PCT Filed: Feb. 15, 2018
(86) PCT No.: PCT/EP2018/053757

§ 371 Date: Aug. 15, 2019

(30) Foreign Application Priority Data
Feb. 20, 2017 (EP) 17156941.1

(51) Int. Cl. A23L 33/18; A23L 33/19; A23L 33/00; A23L 33/135; A61K 38/01

(52) U.S. Cl. CPC A23L 33/18; A23L 33/19; A23V 2002/00; A23L 33/135; A61K 38/018; A23L 33/40

(57) ABSTRACT

The present invention relates to a nutritional composition comprising from 1.5 to 2.3 g/100 kcal of partially hydrolysed whey proteins for use in infants or young children in promoting a glucose and/or an insulin response(s) that is/are similar to the glucose and/or insulin response(s) of infants or young children fed predominantly or exclusively with human breast milk or with intact proteins.

### FIELD OF THE INVENTION

The present invention relates to nutritional compositions for infants or young children and their health effects. In particular, it relates to infant formula comprising partially hydrolysed proteins for inducing a glucose and an insulin responses that are close/similar to the ones of infants fed predominantly or exclusively with human breast milk (HBM).

### BACKGROUND OF THE INVENTION

Evidence suggests that infancy may be a critical period in the programming of metabolic disorders. Mother's milk is recommended for all infants. However, in some cases breast feeding is inadequate or unsuccessful for medical reasons or the mother chooses not to breast feed. Nutritional compositions such as infant formula have been developed for these situations.

Nutritional compositions for infants and young children are often sold as powders to be reconstituted with water or in some instances as ready to drink or concentrated liquid compositions. Those compositions are intended to cover most or all the nutritional needs of the infants or young children.

It is known however, that human breast milk represents the ultimate gold standard in terms of infants' nutrition. Infant formula manufacturers have therefore made many attempts to induce nutritional health effects close to or similar to the benefits of human breast milk. However many studies have shown that infant formula do not induce the identical effects on the body compared to human breast milk.

For example, some studies have shown that infants fed with infant formulas have generally higher insulin secretion than the breastfed ones (Lucas A et al. Acta Paediatric. Scand. 1981; 70: 195-200). Infant formula fed infants may therefore be more prone to develop insulin resistance or diabetes later in life. It has indeed been established that a diet which induces low glycemic and insulineamic responses after the consumption, has advantageous effect on the risk factors for type 2 diabetes and cardiovascular disease (Frost G, Leeds A A, Doré C J, Madeiros S, Brading S, Dornhorst A. Glycaemic index as a determinant of serum HDL-cholesterol concentration. Lancet 1999; 353:1045-8; McKeown N M, Meigs J B, Liu S, Saltzman E, Wilson P W, Jacques P F. Carbohydrate nutrition, insulin resistance, and the prevalence of the metabolic syndrome in the Framingham Offspring Cohort. Diabetes care 2004; 27:538-46). High postprandial blood glucose, together with an elevated related hyperinsulinemia has been implicated in the development of chronic metabolic diseases like obesity, type 2 diabetes mellitus (T2DM) and cardiovascular disease (CVD) (Blaak E E et al. Impact of postprandial glycaemia on health and prevention of disease; Obes Rev. 2012; 13(10):923-84).

Hyperglycemia and hyperinsulinemia are both involved in insulin resistance (Mirmiran P. et al., Journal of diabetes and metabolic disease 2016, 15-32) and the link between a high glycemic index (GI) diet and diabetes relates not only to elevated postprandial blood glucose peaks but also an increase in the insulin demand (Augustin L S, Franceschi S, Jenkins D J, Kendall C W, La Vecchia C. Glycemic index in chronic disease: a review. Eur J Clin Nutr 2002; 56:1049-71).

Reduction of post-prandial blood glucose responses is considered by EFSA as a beneficial physiological effect as long as insulin responses are not disproportionally increased (EFSA Journal 2012;10(3):2604).

There is a linear correlation between the glycemic index (GI) and insulinemic index (II) for many carbohydrate rich foods (Björck I M E, et al., Low glycaemic-index foods. Br J Nutr 2000; 83:S149-55). However, milk is known to promote a disproportionally higher insulin response, relative to its low glucose response.

It was shown that the insulin response to milk does not relate solely to the lactose content of milk but it is also triggers by milk proteins (östman E, et al., 2001, Inconsistency between glycemic and insulinemic responses to regular and fermented milk products. Am J Clin Nutr 74,96-100, and Nilsson M, et al., Glycemia and insulinemia in healthy subjects after lactose equivalent meals of milk and other food proteins: the role of plasma amino acids and incretins; AmJ Clin Nutr. 2004; 80:1246-53). The type and nature of proteins has especially been investigated. Proteins can be intact (i.e. non hydrolysed) or hydrolysed (partially or extensively). Hydrolysed proteins may advantageously be used in infant formula, especially in the hypoallergenic ones to avoid cow's milk allergy. However it was reported that protein hydrolysate stimulate insulin secretion to a higher extent than intact protein (Calbet J A, MacLean D A. Plasma glucagon and insulin responses depend on the rate of appearance of amino acids after ingestion of different protein solutions in humans; J Nutr 2002; 132:2174-82).

Infant formula can also comprise whey and/or casein proteins. Whey proteins are particularly privileged over casein proteins in infant formula to be closer with breast milk that is whey predominant. They are also known to especially enhance immune function, have antioxidant activity, and a faster absorption. However whey proteins are more insulogenic than casein proteins, as especially illustrated in WO2010/112430, in Acheson K J, et al. Protein choices targeting thermogenesis and metabolism; Am J Clin Nutr 2011; 93:525-34 and in Pouteau E. Opti-Prot Clinical trial—Summary report; R&D report 2011, RDLS110081.

Some infant formulas that especially target infants at risk of developing cow's milk allergy and/or atopic disease, contain 100% whey in the form of partially hydrolyzed protein (e.g., NAN HA from Nestle). The benefit of these formulas on the prevention of eczema has been well documented (Von Berg A et al. Allergy. 2016; 71(2): 210-9). However such formulas, because of their hydrolyzed whey protein content, could be more insulinogenic than breast milk or than a standard formula (non hydrolyzed whey/casein formula).

There is therefore a need for infants and young children to promote a nutritional composition which induces glucose and/or insulin response(s) that is/are close to the ones of infants fed predominantly or exclusively with human breast milk, whereas in the same time providing suitable other benefits (like anti-allergy, immunity . . . ).

There is a need to provide infants or young children with the best nutrition that enables to promote a glucose and/or an insulin response(s) that is/are similar to the one(s) of infants or young children fed predominantly or exclusively with human breast milk, said benefits being at short term (i.e. during the nutritional intervention) and/or at long term (i.e. after the nutritional intervention), especially in view of reducing the risk of metabolic disorders later in life.

There is a need to moderate for the sub-normal glucose and/or insulin response(s) observed in non-breast-fed infants or young children.

There is a need to deliver such health benefits in these infants or young children in a manner that does not induce side effects and/or in a manner that is easy to deliver, and well accepted by the parents or health care practitioners.

### SUMMARY OF THE INVENTION

The object of this invention is therefore to provide an infant formula especially adapted to infants at risk of developing cow's milk allergy and/or atopic disease but having a glucose and insulin responses similar to that observed with breast milk or with a standard formula (e.g. Whey/casein ratio of 70/30 in intact form).

The present inventors have surprisingly found that a nutritional composition comprising from 1.5 to 2.3 g/100 kcal of partially hydrolysed whey proteins is especially interesting to promote a glucose and an insulin responses that are similar to the glucose and insulin responses of individuals fed predominantly or exclusively with human breast milk or with intact proteins. It especially prevents any significant increase of the glucose and insulin responses in comparison to the responses obtained for individuals fed predominantly or exclusively with human breast milk or with intact proteins.

The present inventors have also found that a nutritional composition comprising from 1.5 to 2.3 g/100 kcal of partially hydrolysed whey proteins is also particularly interesting for use in promoting and/or inducing a glucose and an insulin responses lower than the responses of individuals fed predominantly or exclusively with a nutritional composition comprising more than 2.3 g/100 kcal (e.g. 2.8 g/100 kcal) of partially hydrolysed whey proteins.

The nutritional composition of the present invention can be especially used in providing a healthy growth, preventing a disorder linked to an increase in insulin secretion and/or preventing insulin resistance and/or diabetes later in life and/or obesity later in life in infants or young children.

### FIGURES

FIG. 1 represents the average profile for insulin with mean and Standard error of mean (SE).

FIG. 2 represents the average profile for glucose with mean and Standard error of mean (SE).

FIG. 3 represents the blood concentration of C-peptide after meal intake.

FIG. 4 represents the postprandial mean increase in the plasma peak values and 2-hour area under the curves (2h-IAUC) of glucose, insulin and C-peptide.

### DETAILED DESCRIPTION OF THE INVENTION

As used herein, the following terms have the following meanings.

The term “infant” means a child under the age of 12 months.

The expression “young child” means a child aged between one and three years, also called toddler.

An “infant or young child born by C-section” means an infant or young child who was delivered by caesarean. It means that the infant or young child was not vaginally delivered.

An “infant or young child vaginally born” means an infant or young child who was vaginally delivered and not delivered by caesarean.

A “preterm” or “premature” means an infant or young child who was not born at term. Generally it refers to an infant or young child born prior to the completion of 37 weeks of gestation.

By the expression “small for gestational age” or “SGA”, it is intended to mean an infant or young child who is smaller in size than normal for their gestational age at birth, most commonly defined as a weight below the 10th percentile for the gestational age. In some embodiments, SGA may be associated with Intrauterine growth restriction (IUGR), which refers to a condition in which a fetus is unable to achieve its potential size.

By the expression “low birth weight”, it should be understood as any body weight under 2500 g at birth.

The expression “nutritional composition” means a composition which nourishes a subject. This nutritional composition is usually to be taken orally or intravenously, and it usually includes a lipid or fat source and a protein source.

In a particular embodiment the nutritional composition of the present invention is a hypoallergenic nutritional composition. The expression “hypoallergenic nutritional composition” means a nutritional composition which is unlikely to cause allergic reactions.

In a particular embodiment the nutritional composition of the present invention is a “synthetic nutritional composition”. The expression “synthetic nutritional composition” means a mixture obtained by chemical and/or biological means, which can be chemically identical to the mixture naturally occurring in mammalian milks (i.e. the synthetic composition is not breast milk).

The expression “infant formula” as used herein refers to a foodstuff intended for particular nutritional use by infants during the first months of life and satisfying by itself the nutritional requirements of this category of person (Article 2(c) of the European Commission Directive 91/321/EEC 2006/141/EC of 22 Dec. 2006 on infant formulae and follow-on formulae). It also refers to a nutritional composition intended for infants and as defined in Codex Alimentarius (Codex STAN 72-1981) and Infant Specialities (incl. Food for Special Medical Purpose). The expression “infant formula” encompasses both “starter infant formula” and “follow-up formula” or “follow-on formula”. In some embodiments, the infant formula is a preterm formula.

A “follow-up formula” or “follow-on formula” is given from the 6th month onwards. It constitutes the principal liquid element in the progressively diversified diet of this category of person.

The expression “baby food” means a foodstuff intended for particular nutritional use by infants or young children during the first years of life.

The expression “infant cereal composition” means a foodstuff intended for particular nutritional use by infants or young children during the first years of life.

The term “fortifier” refers to liquid or solid nutritional compositions suitable for mixing with breast milk or infant formula.

The nutritional composition of the present invention can be in solid form (e.g. powder) or in liquid form. The amount of the various ingredients (e.g. the proteins) can be expressed in g/100 g of composition on a dry weight basis when it is in a solid form, e.g. a powder, or as a concentration in g/L of the composition when it refers to a liquid form (this latter also encompasses liquid composition that may be obtained from a powder after reconstitution in a liquid such as milk, water . . . , e.g. a reconstituted infant formula or a follow-on/follow-up formula or an infant cereal product or any other formulation designed for infant nutrition). They can also be expressed in g/100 kcal.

The “mother's milk” should be understood as the breast milk or colostrum of the mother. HBM refers to Human Breast Milk. HBM and BM can be used alternatively.

The expressions “infants/young children fed exclusively with human breast milk”, “infants or young children exclusively breast fed”, “exclusive breast fed infants or young children” and “breast-fed infants/young children” can be used interchangeably. They refer to infants or young children fed with a great majority (i.e. at least 90%, or at least 95%, or at least 99%) or all (100%) of nutrients and/or energy originating from human breast milk.

The expression “infants or young children exclusively fed” a certain composition refers to infants or young children fed with a great majority (i.e. at least 90%, or at least 95%, or at least 99%) or all (100%) of nutrients and/or energy originating from this certain composition.

The expression “infants or young children predominantly fed” a certain composition refers to infants or young children fed with nutritional sources of nutrients and/or energy predominantly originating from this certain composition. Predominantly refers to at least 50% (or at least 60% or at least 75%) of those nutrients and/or energy, such as from 50% to 90%, or from 60% to 80%.

By the expressions “preventing” or “prevention”, it is meant avoiding that a physical state, a condition or their consequences occurs and/or decreasing its incidence (i.e. reduction of the frequency) and/or decreasing the risk that a physical state, a condition or their consequences occurs.

The prevention of a physical state, a condition or their consequences can occur during the treatment (i.e. during the administration of the composition of the present invention, either immediately after the start of the administration or some time after, e.g. some days or weeks after the start). But it can also encompass the prevention later in life. The term “later in life” encompasses the effect after the termination of the intervention or treatment. The effect “later in life” can be from 1 week to several years, such as from several weeks to several months, for example from 2 to 4 weeks, from 2 to 6 weeks, from 2 to 8 weeks, from 1 to 6 months, from 2 to 12 months, from 2 to 15 years, or from 3 to 8 years.

The expression “a glucose and/or an insulin response(s) that is/are similar to” means that the response(s) is/are not statistically different or not significantly different. In a particular embodiment, it especially means that the glucose and/or insulin response(s) is/are lower but not statistically different.

The terms “hydrolysed” and “hydrolyzed” can be used interchangeably, as well as “hydrolysate” and “hydrolyzate”.

The terms “proteins” and “protein” can be used interchangeably.

The term “prebiotic” means non-digestible carbohydrates that beneficially affect the host by selectively stimulating the growth and/or the activity of healthy bacteria such as bifidobacteria in the colon of humans (Gibson G R, Roberfroid M B. Dietary modulation of the human colonic microbiota: introducing the concept of prebiotics. J Nutr. 1995; 125:1401-12).

The term “probiotic” means microbial cell preparations or components of microbial cells with a beneficial effect on the health or well-being of the host. (Salminen S, Ouwehand A. Benno Y. et al. “Probiotics: how should they be defined” Trends Food Sci. Technol. 1999:10 107-10). The microbial cells are generally bacteria or yeasts.

The term “cfu” should be understood as colony-forming unit.

All percentages are by weight unless otherwise stated.

In addition, in the context of the invention, the terms “comprising” or “comprises” do not exclude other possible elements. The composition of the present invention, including the many embodiments described herein, can comprise, consist of, or consist essentially of the essential elements and limitations of the invention described herein, as well as any additional or optional ingredients, components, or limitations described herein or otherwise depending on the needs.

Any reference to prior art documents in this specification is not to be considered an admission that such prior art is widely known or forms part of the common general knowledge in the field.

The invention will now be described in further details. It is noted that the various aspects, features, examples and embodiments described in the present application may be compatible and/or combined together.

An object of the present invention is therefore a nutritional composition comprising from 1.5 to 2.3 g/100 kcal of partially hydrolysed whey proteins for use in infants or young children (especially those at risk of allergy) in promoting a glucose and/or an insulin response(s) that is/are similar to the glucose and/or insulin response(s) of infants or young children fed exclusively with human breast milk.

The present invention is also about a nutritional composition comprising from 1.5 to 2.3 g/100 kcal of partially hydrolysed whey proteins for use in infants or young children in promoting a glucose and/or an insulin response(s) that is/are similar to the glucose and/or insulin response(s) of infants or young children fed exclusively with intact proteins.

This nutritional composition of the present invention can be used to prevent any significant increase of the glucose and/or insulin response(s) in comparison to the response(s) obtained for infants or young children fed exclusively with human breast milk or with intact proteins.

As previously indicated, the nutritional composition of the present invention is for use in infants or young children in promoting a glucose and/or an insulin response(s) that is/are similar to the response(s) of infants or young children fed exclusively with human breast milk or with intact proteins. By similarity, it means that the response(s) is/are lower, equal or (slightly) higher but not statistically different or not significantly different. This may be defined by the following values:
the nutritional composition of the invention provides a mean increase in the glucose and insulin responses that is not more than 35% lower to the glucose and insulin responses of infants or young children fed predominantly or exclusively with human breast milk or with intact proteins, (based on 2 h IAUC; 2 hours incremental area under curves), such as not more than 30% lower in glucose response (e.g. 29%, 10% . . . ) or not more than 15% (e.g. 8%, 2.6%) lower in insulin response.
the nutritional composition of the invention also provides a mean increase in the glucose and insulin responses that is not more than 15% higher to the glucose and insulin responses of infants or young children fed predominantly or exclusively with human breast milk or with intact proteins (based on 2 h IAUC; 2 hours incremental area under curves), such as not more than 10% higher in glucose and/or insulin response(s). These % values are lower than in the previous paragraph since having lower responses is rather positive whereas having higher responses could be more detrimental as it increases the risks for the infants/young children to develop for example metabolic health disorders (disorder linked to an increase in insulin secretion, insulin resistance, diabetes later in life, obesity later in life . . . ).

The nutritional composition of the present invention can also be for use in promoting and/or inducing in infants or young children a glucose and/or an insulin response(s) that is/are lower than the glucose and/or insulin response(s) of infants or young children fed predominantly or exclusively with a nutritional composition comprising more than 2.3 g/100 kcal (e.g. 2.8 g/100 kcal) of partially hydrolysed whey proteins.

This nutritional composition indeed provides a mean increase in glucose and insulin responses (2-h IAUC) that is lower by at least 35% than the glucose and insulin responses of infants or young children fed predominantly or exclusively with a nutritional composition comprising more than 2.3 g/100 kcal of partially hydrolysed whey proteins, such as at least 50% lower for glucose response or at least 35% lower for insulin response.

The different objects of the present invention are unitary in the sense that they allow promoting a glucose and/or an insulin response(s) in infants or young children that is/are beneficial for said infants or young children.

The nutritional composition according to the invention contains a protein source. The protein are in an amount of from 1.5 to 2.3 g per 100 kcal. In some embodiments, the protein amount can be from 1.6 to 2.2 g per 100 kcal, such as from 1.7 to 2.1 g per 100 kcal, or below 2.0 g per 100 kcal, e.g. between 1.8 to 2.0 g/100 kcal. In some embodiments the protein amount is from 1.9 to 2.1 g protein per 100 kcal.

The nutritional composition according to the invention contains whey proteins. There may also be caseins, but in some preferred embodiments, 100% of the proteins are whey proteins. The protein source may be based on acid whey or sweet whey or mixtures thereof and may include alpha-lactalbumin and beta-lactoglobulin in any desired proportions. “Alpha-Lactalbumin” refers to a high-quality, easy-to-digest whey protein that comprises 20-25% of total human breast milk (HBM) protein and is the primary protein found in HBM. The structure of alpha-lactalbumin is comprised of 123 amino acids and 4 disulfide bridges and the protein has a molecular weight of 14.2K Daltons. Alpha-lactalbumin is ideal for lower protein infant formulas due to its high content of essential amino acids, particularly tryptophan. In one embodiment, the nutritional composition of this invention comprises alpha-lactalbumin in an amount of from about 0.2 to about 0.4 g/100 kcal of the nutritional composition, or in an amount of at least 1.7 g/L, or at least 2.0 g/L or at least 2.3 g/L, or at least 2.6 g/L of the nutritional composition.

The whey proteins (preferably all or at least a part thereof) present in the nutritional composition are partially hydrolysed.

The degree of hydrolysis is typically between 2 and 20%. Hydrolysed proteins are especially beneficial, for example for infants or young children believed to be at risk of developing cow's milk allergy. The hydrolysis process may be carried out as desired and as is known in the art. The partially hydrolysed whey proteins may be concentrated and/or extracted from natural sources such as milk (e.g. bovine milk) or milk fractions which have been preferably modified, treated to, for example, produce or concentrate the partially hydrolysed whey proteins of the invention.

The enzymatic hydrolysis may be carried out using any of a whey starting material containing whey proteins or a combination thereof. This starting material may be a whey from cheese making, particularly a sweet whey such as that resulting from the coagulation of casein by rennet, or an acidic whey from the coagulation of casein by an acid, or the acidifying ferments, or even a mixed whey resulting from coagulation by an acid and by rennet. This starting material may be whey demineralized by ion exchange and/or by electrodialysis. This whey may be a concentrate of whey proteins more or less free from lactose obtained, for example, by ultrafiltration optionally followed by dialysis. If the whey fraction used as the starting material is substantially lactose free, it is found that the protein suffers much less lysine blockage during the hydrolysis process. This enables the extent of lysine blockage to be reduced from about 15% by weight of total lysine to less than about 10% by weight of lysine; for example about 7% by weight of lysine which greatly improves the nutritional quality of the protein source. The starting material may even be a combination of the above-mentioned starting materials and lactose. It may be in the form of a true or colloidal aqueous solution or in the form of a powder. In the latter case, the powder is dissolved in preferably demineralized water to form an aqueous solution. The whey protein source can also be obtained via the process described in U.S. Pat. No. 6,787,158.

For example, the partially hydrolysed whey protein may be prepared by enzymatically hydrolysing the whey fraction in one or more steps. For example, they may be produced using the method disclosed in U.S. Pat. No. 5,039,532A. The enzymatic hydrolysis process includes a two-step hydrolysis reaction with a heat denaturation step in between to ensure that the final hydrolysate is substantially free of intact allergenic proteins.

The enzymes used for the hydrolysis may be for example trypsine, such as from porcine origin, and/or alcalase, especially when a product with halal status is desired.

In a particular embodiment, the hydrolysis process is a 2 steps hydrolysis using a batch process for protein breakdown. Protein sources can be for example Modified Sweet Whey proteins/Demineralized Whey Proteins (83/17 protein ratio) or Whey Protein Concentrate 35. The enzyme is a protease that can be porcine Trypsin, for example sold by Novozymes or by Bioseutica, or an alcalase like microbial E, sold by Novozymes.

After partial hydrolysis is performed by methods known by the skilled person, the enzyme is preferably inactivated.

The preliminary hydrolysis may be carried out after a pH adjustment to 7-8 with an aqueous solution of a base (e.g. KOH/NaOH/Ca(OH)2) and addition of 0.1-3% of an enzyme (e.g. trypsin or alcalase) for 1.5 h to 5 hours at 40-60° C. This hydrolysis may be carried out in a reactor or, alternatively, in a tube. The hydrolysate is then subjected to a heat treatment at 80° to 100° C. for 3 to 10 minutes at a pH value of 7 to 8. The heat treatment time and temperature are of course interrelated, the lower temperature limit corresponding to the upper time limit and vice versa. In industrial heat exchangers, a temperature of approximately 95° C. and a residence time of the order of 5 minutes have proved to be sufficient for denaturing the proteins. It has, in effect, been found that denaturing these proteins makes them accessible to the subsequent enzymatic degradation. It is advisable to mention that the heat treatment inactivates the enzyme. The hydrolysate is then cooled to a temperature of 40° to 60° C. and preferably to a temperature of approximately 55° C., which is the optimal temperature for the hydrolytic activity. The pH value is preferably adjusted to 7-7.5 by addition of an aqueous solution of a base (e.g. KOH/NaOH/Ca(OH)2). After addition of 0.4-5% of the proteolytic enzyme (e.g. trypsin or alcalase) in aqueous solution, the hydrolysis is carried out for 1.5 to 3.5 h in batch process. In some other embodiments, the second hydrolysis may alternatively be done continuously (tubes).

Irrespective of the mode employed (batch or continuous) selected for the second hydrolysis, the hydrolysed product then undergoes a heat treatment which inactivates the enzyme. This heat treatment comprises heating the hydrolysate to a temperature of 75-90° C. depending on the enzyme and keeping it at that temperature for about 5 minutes to promote auto-digestion of the enzyme. This treatment advantageously being followed by sterilization, preferably at ultra-high temperature, for example at 125° to 135° C., for 2 to 3 minutes by injection of steam or in a heat exchanger. The hydrolysate may then be dried, for example by spray drying or by freeze drying for different applications, or may even be subsequently treated. In the latter case, the enzyme may be inactivated during the subsequent treatment.

In a particular embodiment the whey proteins of the nutritional composition of the present invention forms a complex hydrolysate.

In a particular embodiment the nutritional composition of the present invention comprises partially hydrolysed whey proteins that have a % amino-N/TN value of from 8 to 17%, such as 10-15%, measured by the TNBS method. The TNBS (trinitro-benzene-sulfonic acid) method is known by the skilled person. It is based on the reaction of primary amino groups with trinitro-benzene-sulfonic acid (TNBS) reagent and it is described in Adler-Nissen J. 1979; Determination of the degree of hydrolysis of food protein hydrolysates by trinitrobenzenesulfonic acid. Agric Food Chem 27:1256-1262, or in Spellman D. et al (2003); International Dairy Journal 13, 447-453.

This method is used to define hydrolysis particularly in case of complex hydrolysate. It corresponds to the percentage of nitrogen present as free amino nitrogen [α- and ε-amino groups]. For comparison of hydrolysates, the amino-N(or TNBS-reactive nitrogen) content is expressed as a percentage of total nitrogen [% amino-N/TN].

In a particular embodiment the nutritional composition of the present invention comprises partially hydrolysed whey proteins that have a % NPN/TN value of 75-95%, such as 80-90%. Indeed the Non Protein Nitrogen content can also be used to assess the extent of protein hydrolysis, especially in case of complex hydrolysate. Non Protein Nitrogen is the nitrogen present in nitrogen compounds, generally of low molecular weight, which remain soluble in a 12% trichloroacetic acid solution. The technique is known by the skilled person and it comprises especially the following steps: dissolution of a test portion is made, then 24% of trichloroacetic acid solution is added so that a final concentration of 12% (w/v) is reached. Filtration is then performed, then nitrogen determination in an aliquot of the filtrate is made. Details can be found in ISO 8968-4:2016 (IDF 20-4).

In a particular embodiment the nutritional composition according to the invention is a hypoallergenic nutritional composition.

The nutritional composition according to the present invention generally contains a carbohydrate source. This is particularly preferable in the case where the nutritional composition of the invention is an infant formula. In this case, any carbohydrate source conventionally found in infant formula such as lactose, sucrose, saccharose, maltodextrin, starch and mixtures thereof may be used although one of the preferred sources of carbohydrates is lactose.

The nutritional composition according to the present invention generally contains a source of lipids. This is particularly relevant if the nutritional composition of the invention is an infant formula. In this case, the lipid source may be any lipid or fat which is suitable for use in infant formula. Some suitable fat sources include palm oil, high oleic sunflower oil and high oleic safflower oil. The essential fatty acids linoleic and α-linolenic acid may also be added, as well small amounts of oils containing high quantities of preformed arachidonic acid and docosahexaenoic acid such as fish oils or microbial oils. The fat source may have a ratio of n-6 to n-3 fatty acids of about 5:1 to about 15:1; for example about 8:1 to about 10:1.

The nutritional composition according to the present invention may also comprise at least an oligosaccharide(s) and/or at least a fiber(s) and/or at least a precursor(s) thereof. The other oligosaccharide and/or fiber and/or precursor thereof may be selected from the list comprising galacto-oligosaccharides (GOS), fructo-oligosaccharides (FOS), inulin, xylooligosaccharides (XOS), polydextrose, fucosylated oligosaccharide(s), N-acetylated oligosaccharide(s), sialylated oligosaccharides, sialic acid, fucose and any combination thereof. They may be in an amount between 0 and 10% by weight of composition.

Suitable commercial products that can be used such as combinations of FOS with inulin such as the product sold by BENEO under the trademark Orafti, or polydextrose sold by Tate & Lyle under the trademark STA-LITE®.

In a particular embodiment, the nutritional composition according to the invention can comprise at least about 0.4 g or at least 0.7 g of oligofructose per 100 kcal of the composition such as from about 0.4 to about 0.9 g, from about 0.4 to about 0.7 g, from about 0.4 to about 0.5 g, from about 0.7 to about 0.8 g, or from about 0.7 to about 0.9 g oligofructose per 100 kcal.

In some embodiments the oligofructose has a degree of polymerization of from 2 to 10. In some embodiments, at least 80%, 90%, 95%, 99% or 100% of the oligofructose has a degree of polymerization of from 2 to 8 (between 2 and 8).

In a particular embodiment, the nutritional composition according to the invention can comprise GOS. A galacto-oligosaccharide is an oligosaccharide comprising two or more galactose molecules which has no charge and no N-acetyl residue. Suitable galacto-oligosaccharides that may also be added in the nutritional composition according to the present invention include Galβ1,3Galβ1,4Glc, Galβ1,6Galβ1,4Glc, Galβ1,3Galβ1,3Galβ1,4Glc, Galβ1,6Galβ1,6Galβ1,4Glc, Galβ1,3Galβ1,6Galβ1,4Glc, Galβ1,6Galβ1,3Galβ1,4Glc, Galβ1,6Galβ1,6Galβ1,6Glc, Galβ1,3Galβ1,3Glc, Galβ1,4Galβ1,4Glc and Galβ1,4Galβ1,4Galβ1,4Glc but also any mixture thereof. Synthesized galacto-oligosaccharides such as Galβ1,6Galβ1,4Glc, Galβ1,6Galβ1,6Galβ1,6Glc, Galβ1,3Galβ1,4Glc, Galβ1,6Galβ1,6Galβ1,4Glc, Galβ1,6Galβ1,3Galβ1,4Glc, Galβ1,3Galβ1,6Galβ1,4Glc, Galβ1,4Galβ1,4Glc and Galβ1,4Galβ1,4Galβ1,4Glc and mixture thereof are commercially available under trademarks Vivinal® and Elix'or®. Other suppliers of oligosaccharides are Dextra Laboratories, Sigma-Aldrich Chemie GmbH and Kyowa Hakko Kogyo Co., Ltd. Alternatively, specific glycotransferases, such as galoctosyltransferases may be used to produce neutral oligosaccharides.

In a particular embodiment, the nutritional composition can also contain at least one bovine milk oligosaccharide. Conventional technologies for fractioning and enriching bovine milk fractions in bovine milk derived oligosaccharides can be used (such conventional technologies include column filtration, resin-filtration, nano-filtration, enzymatic treatment specially with beta-galactosidase, precipitation of proteins, crystallisation and separation of lactose etc, . . . ). Some fractions of bovine milk enriched in oligosaccharides are commercially available or have been described for example in EP2526784 A1.

In a particular embodiment, the nutritional composition may also additionally comprise an oligosaccharide mixture (“BMOS”) that comprises from 0.1 to 4.0 wt % of N-acetylated oligosaccharide(s), from 92.0 to 98.5 wt % of the galacto-oligosaccharide(s) and from 0.3 to 4.0 wt % of the sialylated oligosaccharide(s).

In a particular embodiment, the nutritional composition according to the invention can comprise fucosylated oligosaccharide(s), N-acetylated oligosaccharide(s) and/or sialylated oligosaccharide(s).

The fucosylated oligosaccharide(s) can be 2FL.

The N-acetylated oligosaccharide(s) can be LNT and/or LNnT.

The sialylated oligosaccharide(s) can be 3′ sialyllactose (3-SL) and/or 6′ sialyllactose (6-SL).

The nutritional composition of the present invention can further comprise at least one probiotic (or probiotic strain), such as a probiotic bacterial strain.

The probiotic microorganisms most commonly used are principally bacteria and yeasts of the following genera: Lactobacillus spp., Streptococcus spp., Enterococcus spp., Bifidobacterium spp. and Saccharomyces spp.

In some particular embodiments, the probiotic is a probiotic bacterial strain. In some specific embodiments, it is particularly Bifidobacteria and/or Lactobacilli.

Suitable probiotic bacterial strains include Lactobacillus rhamnosus ATCC 53103 available from Valio Oy of Finland under the trademark LGG, Lactobacillus rhamnosus CGMCC 1.3724, Lactobacillus paracasei CNCM I-2116, Lactobacillus johnsonii CNCM 1-1225, Streptococcus salivarius DSM 13084 sold by BLIS Technologies Limited of New Zealand under the designation KI2, Bifidobacterium lactis CNCM 1-3446 sold inter alia by the Christian Hansen company of Denmark under the trademark Bb 12, Bifidobacterium longum ATCC BAA-999 sold by Morinaga Milk Industry Co. Ltd. of Japan under the trademark BB536, Bifidobacterium breve sold by Danisco under the trademark Bb-03, Bifidobacterium breve sold by Morinaga under the trade mark M-16V, Bifidobacterium infantis sold by Procter & Gamble Co. under the trademark Bifantis and Bifidobacterium breve sold by Institut Rosell (Lallemand) under the trademark R0070.

In a particular embodiment the probiotic is a Bifidobacterium lactis, such as Bifidobacterium lactis CNCM 1-3446.

The nutritional composition according to the invention may contain from 10e3 to 10e12 cfu of probiotic strain, more preferably between 10e7 and 10e12 cfu such as between 10e8 and 10e10 cfu of probiotic strain per g of composition on a dry weight basis.

In one embodiment the probiotics are viable. In another embodiment the probiotics are non-replicating or inactivated. There may be both viable probiotics and inactivated probiotics in some other embodiments.

The nutritional composition of the invention can further comprise at least one phage (bacteriophage) or a mixture of phages, preferably directed against pathogenic Streptococci, Haemophilus, Moraxella and Staphylococci.

The nutritional composition according to the invention can be for example an infant formula, a starter infant formula, a follow-on or follow-up formula, a preterm formula, a baby food, an infant cereal composition, a fortifier such as a human milk fortifier, or a supplement. In some particular embodiments, the composition of the invention is an infant formula, a fortifier or a supplement that may be intended for the first 4 or 6 months of age. In a preferred embodiment the nutritional composition of the invention is an infant formula.

In some other embodiments the nutritional composition of the present invention is a fortifier. The fortifier can be a breast milk fortifier (e.g. a human milk fortifier) or a formula fortifier such as an infant formula fortifier or a follow-on/follow-up formula fortifier.

When the nutritional composition is a supplement, it can be provided in the form of unit doses.

The nutritional composition of the present invention can be in solid (e.g. powder), liquid or gelatinous form.

The nutritional composition of the invention may also contain all vitamins and minerals understood to be essential in the daily diet and in nutritionally significant amounts. Minimum requirements have been established for certain vitamins and minerals. Examples of minerals, vitamins and other nutrients optionally present in the composition of the invention include vitamin A, vitamin B1, vitamin B2, vitamin B6, vitamin B12, vitamin E, vitamin K, vitamin C, vitamin D, folic acid, inositol, niacin, biotin, pantothenic acid, choline, calcium, phosphorous, iodine, iron, magnesium, copper, zinc, manganese, chlorine, potassium, sodium, selenium, chromium, molybdenum, taurine, and L-carnitine. Minerals are usually added in salt form. The presence and amounts of specific minerals and other vitamins will vary depending on the intended population.

If necessary, the nutritional composition of the invention may contain emulsifiers and stabilisers such as soy, lecithin, citric acid esters of mono- and diglycerides, and the like.

The nutritional composition of the invention may also contain other substances which may have a beneficial effect such as lactoferrin, nucleotides, nucleosides, and the like.

The nutritional composition of the invention may also contain carotenoid(s).

The nutritional composition according to the invention may be prepared in any suitable manner. A composition will now be described by way of example.

For example, a formula such as an infant formula may be prepared by blending together the protein source (constituted or comprising the partially hydrolyzed whey proteins that may be obtained as previously described), the carbohydrate source and the fat source in appropriate proportions. If used, the emulsifiers may be included at this point. The vitamins and minerals may be added at this point but they are usually added later to avoid thermal degradation. Any lipophilic vitamins, emulsifiers and the like may be dissolved into the fat source prior to blending. Water, preferably water which has been subjected to reverse osmosis, may then be mixed in to form a liquid mixture. The temperature of the water is conveniently in the range between about 50° C. and about 80° C. to aid dispersal of the ingredients. Commercially available liquefiers may be used to form the liquid mixture.

The liquid mixture is then homogenised, for example in two stages.

The liquid mixture may then be thermally treated to reduce bacterial loads, by rapidly heating the liquid mixture to a temperature in the range between about 80° C. and about 150° C. for a duration between about 5 seconds and about 5 minutes, for example. This may be carried out by means of steam injection, an autoclave or a heat exchanger, for example a plate heat exchanger.

Then, the liquid mixture may be cooled to between about 60° C. and about 85° C. for example by flash cooling. The liquid mixture may then be again homogenised, for example in two stages between about 10 MPa and about 30 MPa in the first stage and between about 2 MPa and about 10 MPa in the second stage. The homogenised mixture may then be further cooled to add any heat sensitive components, such as vitamins and minerals. The pH and solids content of the homogenised mixture are conveniently adjusted at this point.

If the final product is to be a powder, the homogenised mixture is transferred to a suitable drying apparatus such as a spray dryer or freeze dryer and converted to powder. The powder should have a moisture content of less than about 5% by weight. The probiotic strain(s) (if used) may be added at this stage by dry-mixing or by blending them in a syrup form of crystals, and the mixture is spray-dried or freeze-dried.

If a liquid composition is preferred, the homogenised mixture may be sterilised then aseptically filled into suitable containers or may be first filled into the containers and then retorted.

In another embodiment, the composition of the invention may be a supplement. The supplement may be in the form of tablets, capsules, pastilles or a liquid for example. The supplement may further contain protective hydrocolloids (such as gums, proteins, modified starches), binders, film forming agents, encapsulating agents/materials, wall/shell materials, matrix compounds, coatings, emulsifiers, surface active agents, solubilizing agents (oils, fats, waxes, lecithins etc.), adsorbents, carriers, fillers, co-compounds, dispersing agents, wetting agents, processing aids (solvents), flowing agents, taste masking agents, weighting agents, jellifying agents and gel forming agents. The supplement may also contain conventional pharmaceutical additives and adjuvants, excipients and diluents, including, but not limited to, water, gelatine of any origin, vegetable gums, lignin-sulfonate, talc, sugars, starch, gum arabic, vegetable oils, polyalkylene glycols, flavouring agents, preservatives, stabilizers, emulsifying agents, buffers, lubricants, colorants, wetting agents, fillers, and the like.

Further, the supplement may contain an organic or inorganic carrier material suitable for oral or parenteral administration as well as vitamins, minerals trace elements and other micronutrients in accordance with the recommendations of Government bodies such as the USRDA.

The nutritional composition according to the invention is for use in infants or young children. It is particularly adapted for infants under 6 months of age.

The infants or young children may be born term or preterm.

In one embodiment, the nutritional composition of the present invention may also be used in an infant or a young child that was born small for gestational age or low birth weight.

Infants or young children with low birth weight may or may not be preterm, and similarly, infants or young children who are small for gestational age may or may not be preterm.

In one embodiment, the nutritional composition of the present invention may also be used in an infant or a young child who suffers of intra uterine growth retardation (IUGR).

The nutritional composition of the present invention may also be used in an infant or a young child that was born by C-section or that was vaginally delivered.

All infants and young children can benefit from the invention as all of them are or can be, at a certain age, susceptible to develop insulin resistance.

In some advantageous embodiments of the invention, the nutritional composition is for use in infants or young children at risk of developing obesity later in life, diabetes later in life and/or insulin resistance.

For example the nutritional composition of the present invention is for use in infants born from mothers who are overweight or obese and/or who suffer of diabetes or insulin resistance.

The nutritional composition of the present invention can be advantageously used in an infant or young child at risk of developing cow's milk allergy and/or atopic disease, due to the presence of partially hydrolysed whey protein.

The nutritional composition can be administered (or given or fed) at an age and for a period that depends on the needs.

In one embodiment, the infants or young children are 0-36 months of age, such as 0-12 months or 0-6 months of age.

In some particular embodiments, the nutritional composition can be an infant formula and may be especially intended for infants between 0 and 12 months of age fed predominantly with infant formula.

In some advantageous embodiments the nutritional composition can be for example given immediately after birth of the infants. The composition of the invention can also be given during the first week of life of the infant, or during the first 2 weeks of life, or during the first 3 weeks of life, or during the first month of life, or during the first 2 months of life, or during the first 3 months of life, or during the first 4 months of life, or during the first 6 months of life, or during the first 8 months of life, or during the first 10 months of life, or during the first year of life, or during the first two years of life or even more. In some particularly advantageous embodiments of the invention, the nutritional composition is given (or administered) to an infant within the first 4 or 6 months of birth of said infant. In some other embodiments, the nutritional composition of the invention is given few days (e.g. 1, 2, 3, 5, 10, 15, 20 . . . ), or few weeks (e.g. 1, 2, 3, 4, 5, 6, 7, 8, 9, 10 . . . ), or few months (e.g. 1, 2, 3, 4, 5, 6, 7, 8, 9, 10 . . . ) after birth.

The nutritional composition of the present invention may be given for some days (1, 2, 3, 4, 5, 6 . . . ), or for some weeks (1, 2, 3, 4, 5, 6, 7, 8 or even more), or for some months (1, 2, 3, 4, 5, 6, 7, 8, 9, 10, 11 or even more), depending on the needs.

In some embodiments the composition according to the invention can be for use before and/or during the weaning period.

In one embodiment the composition of the invention is given to the infant or young child as a supplementary composition to the mother's milk. In some embodiments the infant or young child receives the mother's milk during at least the first 2 weeks, or the first 1, 2, 4, or 6 months. In one embodiment the nutritional composition of the invention is given to the infant or young child after such period of mother's nutrition, or is given together with such period of mother's milk nutrition. In another embodiment the composition is given to the infant or young child as the sole or primary nutritional composition during at least one period of time, e.g. after the 1st, 2nd or 4th month of life, during at least 1, 2, 4 or 6 months.

In one embodiment the nutritional composition of the invention is a complete nutritional composition (fulfilling all or most of the nutritional needs of the subject). In another embodiment the nutrition composition is a supplement or a fortifier intended for example to supplement human milk or to supplement an infant formula or a follow-on/follow-up formula.

The major and surprising health benefit of the nutritional composition of the present invention is that it allows promoting a glucose and/or an insulin response(s) that is/are similar to the glucose and/or insulin response(s) of infants or young children fed predominantly or exclusively with human breast milk or also with intact proteins, despite the presence of partially hydrolysed whey proteins.

This has the effect to prevent any significant increase of the glucose and/or insulin response(s) in comparison to the response(s) obtained for infants or young children fed predominantly or exclusively with human breast milk or with intact proteins.

The nutritional composition can also be used in promoting and/or inducing in infants or young children a glucose and/or an insulin response(s) that is/are lower than the glucose and/or insulin response(s) of infants or young children fed predominantly or exclusively with a nutritional composition comprising more than 2.3 g/100 kcal (e.g. 2.3-3.2 g/100 kcal, such as 2.5-3.0 g/100 kcal, e.g. 2.8 g/100 kcal) of partially hydrolysed whey proteins.

The risk of getting a disorder linked to an increase in insulin secretion and/or of getting insulin resistance and/or diabetes later in life in infants or young children is prevented whereas partially hydrolysed whey proteins are used and a higher risk was expected. This allows getting the other benefits of partially hydrolysed whey proteins (like the diminution of the risk of allergy and/or atopic disease) without getting any negative consequences on insulin secretion.

Having a hormonal profile closer to breastfed infants allows reducing the risk of obesity and type-2 diabetes later in life.

The nutritional composition according to the present invention can therefore be for use in providing a healthy growth, preventing a disorder linked to an increase in insulin secretion and/or preventing insulin resistance and/or diabetes later in life and/or obesity later in life in infants or young children.

The beneficial health benefits provided by the composition of the invention can be short term and/or long term effects.

The effect may be immediate with the administration of the composition of the present invention, or later in life, i.e. after the administration of the composition, e.g. from 1 week to several years, e.g. from some weeks to some months, for example from 2 to 4 weeks, from 2 to 6 weeks, from 2 to 8 weeks, from 1 to 6 months, from 2 to 12 months, or from 2 to 15 years, or from 3 to 8 years after said administration.

Other Objects:

Another object of the present invention is the use of 1.5 to 2.3 g/100 kcal of partially hydrolysed whey proteins in the preparation of a nutritional composition for promoting a glucose and/or an insulin response(s) that is/are similar to the glucose and/or insulin response(s) of infants or young children fed predominantly or exclusively with human breast milk.

Another object of the present invention is the use of 1.5 to 2.3 g/100 kcal of partially hydrolysed whey proteins in the preparation of a nutritional composition for promoting a glucose and/or an insulin response(s) that is/are similar to the glucose and/or insulin response(s) of infants or young children fed predominantly or exclusively with intact proteins.

Another object of the present invention is the use of 1.5 to 2.3 g/100 kcal of partially hydrolysed whey proteins in the preparation of a nutritional composition for preventing any significant increase of the glucose and/or insulin response(s) in comparison to the response(s) obtained for infants or young children fed predominantly or exclusively with human breast milk.

Another object of the present invention is the use of 1.5 to 2.3 g/100 kcal of partially hydrolysed whey proteins in the preparation of a nutritional composition for preventing any significant increase of the glucose and/or insulin response(s) in comparison to the response(s) obtained for infants or young children fed predominantly or exclusively with intact proteins.

Another object of the present invention is the use of 1.5 to 2.3 g/100 kcal of partially hydrolysed whey proteins in the preparation of a nutritional composition for promoting and/or inducing in infants or young children a glucose and/or an insulin response(s) that is/are lower than the glucose and/or insulin response(s) of infants or young children fed predominantly or exclusively with a nutritional composition comprising more than 2.3 g/100 kcal of partially hydrolysed whey proteins.

Another object of the present invention is a pharmaceutical composition comprising 1.5 to 2.3 g/100 kcal of partially hydrolysed whey proteins for promoting a glucose and/or an insulin response(s) that is/are similar to the glucose and/or insulin response(s) of infants or young children fed predominantly or exclusively with human breast milk.

Another object of the present invention is a pharmaceutical composition comprising 1.5 to 2.3 g/100 kcal of partially hydrolysed whey proteins for promoting a glucose and/or an insulin response(s) that is/are similar to the glucose and/or insulin response(s) of infants or young children fed predominantly or exclusively with intact proteins.

Another object of the present invention is a pharmaceutical composition comprising 1.5 to 2.3 g/100 kcal of partially hydrolysed whey proteins for preventing any significant increase of the glucose and/or insulin response(s) in comparison to the response(s) obtained for infants or young children fed predominantly or exclusively with human breast milk.

Another object of the present invention is a pharmaceutical composition comprising 1.5 to 2.3 g/100 kcal of partially hydrolysed whey proteins for preventing any significant increase of the glucose and/or insulin response(s) in comparison to the response(s) obtained for infants or young children fed predominantly or exclusively with intact proteins.

Another object of the present invention is a pharmaceutical composition comprising 1.5 to 2.3 g/100 kcal of partially hydrolysed whey proteins for promoting and/or inducing in infants or young children a glucose and/or an insulin response(s) that is/are lower than the glucose and/or insulin response(s) of infants or young children fed predominantly or exclusively with a nutritional composition comprising more than 2.3 g/100 kcal of partially hydrolysed whey proteins.

Another object of the present invention refers to a method for promoting a glucose and/or an insulin response(s) that is/are similar to the glucose and/or insulin response(s) of infants or young children fed predominantly or exclusively with human breast milk, said method comprising administering to said infant or young child a nutritional composition comprising 1.5 to 2.3 g/100 kcal of partially hydrolysed whey proteins.

Another object of the present invention refers to a method for promoting a glucose and/or an insulin response(s) that is/are similar to the glucose and/or insulin response(s) of infants or young children fed predominantly or exclusively with intact proteins, said method comprising administering to said infant or young child a nutritional composition comprising 1.5 to 2.3 g/100 kcal of partially hydrolysed whey proteins.

Another object of the present invention refers to a method for preventing any significant increase of the glucose and/or insulin response(s) in comparison to the response(s) obtained for infants or young children fed predominantly or exclusively with human breast milk, said method comprising administering to said infant or young child a nutritional composition comprising 1.5 to 2.3 g/100 kcal of partially hydrolysed whey proteins.

Another object of the present invention refers to a method for preventing any significant increase of the glucose and/or insulin response(s) in comparison to the response(s) obtained for infants or young children fed predominantly or exclusively with intact proteins, said method comprising administering to said infant or young child a nutritional composition comprising 1.5 to 2.3 g/100 kcal of partially hydrolysed whey proteins.

Another object of the present invention refers to a method for promoting and/or inducing in infants or young children a glucose and/or an insulin response(s) that is/are lower to the glucose and/or insulin response(s) of infants or young children fed predominantly or exclusively with a nutritional composition comprising more than 2.3 g/100 kcal of partially hydrolysed whey proteins, said method comprising administering to said infant or young child a nutritional composition comprising 1.5 to 2.3 g/100 kcal of partially hydrolysed whey proteins

The previously-mentioned embodiments and examples (e.g. related to the nutritional composition, the administration, the targeted population . . . ) also apply for these various objects (i.e. uses, pharmaceutical composition, methods . . . ).

### EXAMPLES

The following examples illustrate some specific embodiments of the composition for use according to the present invention. The examples are given solely for the purpose of illustration and are not to be construed as limitations of the present invention, as many variations thereof are possible without departing from the spirit of the invention.

### Example 1

An example of the composition of a nutritional composition (e.g. an infant formula) according to the present invention is given in the below table 1. This composition is given by way of illustration only.

TABLE 1
An example of the composition of a nutritional composition
(e.g. an infant formula) according to the present invention

| Nutrients | per 100 kcal | per litre |
|---|---|---|
| Energy (kcal) | 100 | 670 |
| Protein (g) | 1.83 | 12.3 |
| (100% being | | |
| partially | | |
| hydrolysed whey | | |
| proteins) | | |
| Fat (g) | 5.3 | 35.7 |
| Linoleic acid (g) | 0.79 | 5.3 |
| α-Linolenic acid (mg) | 101 | 675 |
| Lactose (g) | 11.2 | 74.7 |
| Minerals (g) | 0.37 | 2.5 |
| Na (mg) | 23 | 150 |
| K (mg) | 89 | 590 |
| Cl (mg) | 64 | 430 |
| Ca (mg) | 62 | 410 |
| P (mg) | 31 | 210 |
| Mg (mg) | 7 | 50 |
| Mn (μg) | 8 | 50 |
| Se (μg) | 2 | 13 |
| Vitamin A (μg RE) | 105 | 700 |
| Vitamin D (μg) | 1.5 | 10 |
| Vitamin E (mg TE) | 0.8 | 5.4 |
| Vitamin K1 (μg) | 8 | 54 |
| Vitamin C (mg) | 10 | 67 |
| Vitamin B1 (mg) | 0.07 | 0.47 |
| Vitamin B2 (mg) | 0.15 | 1.0 |
| Niacin (mg) | 1 | 6.7 |
| Vitamin B6 (mg) | 0.075 | 0.50 |
| Folic acid (μg) | 9 | 60 |
| Pantothenic acid (mg) | 0.45 | 3 |
| Vitamin B12 (μg) | 0.3 | 2 |
| Biotin (μg) | 2.2 | 15 |
| Choline (mg) | 10 | 67 |
| Fe (mg) | 1.2 | 8 |
| I (μg) | 15 | 100 |
| Cu (mg) | 0.06 | 0.4 |
| Zn (mg) | 0.75 | 5 |

### Example 2

Description of the Clinical Study

The proposed study is a randomized, double blind, monocentre, cross-over study with four study products.

Product 1: Breast milk (BM group)

Product 2: intact protein formula (70% whey/30% casein at 1.88 g protein/100 kcal) (INTACT group)

Product 3: partially hydrolyzed whey protein (1.96 g/100 kcal) (PHw group)

Product 4: Discharge formula for premature babies partially hydrolyzed whey protein at higher concentration (2.8 g/100 kcal) (DISCH group)

See below table 2 for the detailed composition of the 3 formula.

34 adults volunteers (males and females) were recruited for this study with a normal body weight (BMI 19-25 kg/m2). It was also chosen to perform the study in adults (and not infants) for ethic reasons. Though the glucose and insulin responses to the tested products in the absolute values may be different between adults and infants, the relative difference between tested products is not likely to be different.

TABLE 2
composition of the tested infant formula

| | | | | Discharge |
|---|---|---|---|---|
| | | Intact | Partially | Partially |
| | Formula | protein | hydrolysed | hydrolysed |
| | Energy density | 63 | 63 | 73 |
| | (kcal/100 ml) | | | |
| Protein | Content | 1.88 | 1.96 | 2.8 |
| | (g/100 kcal) | | | |
| | Content (g/l) | 11.8 | 12.35 | 20.4 |
| | % whey | 70.0 | 100 | 100 |
| | % casein | 30.0 | | |
| Carbohydrate | Lactose (% total | 100 | 100 | 70 |
| | carbohydrate) | | | |
| | Maltodextrin | | | 30 |
| | (% total | | | |
| | carbohydrate) | | | |
| | Content | 11.6 | 11.6 | 10.5 |
| | (g/100 kcal) | | | |
| | Content (g/l) | 73.1 | 73.08 | 76.6 |
| Lipid | Content | 5.1 | 5.08 | 5.2 |
| | (g/100 kcal) | | | |
| | Content (g/l) | 32.1 | 32.0 | 38 |
| Probiotics | Type | B. lactis | B. lactis | B. lactis |
| Minerals | Na (mg) | 28 | 28 | 35 |
| (/100 kcal) | K (mg) | 100 | 100 | 105 |
| | Cl (mg) | 69.0 | 69 | 75.0 |
| | Ca (mg) | 75.0 | 75 | 110.0 |
| | P (mg) | 42.0 | 42 | 65.0 |
| | Mg (mg) | 7.6 | 7.6 | 9.9 |
| | Mn (μg) | 10.0 | 10.8 | 16.0 |
| | Ca/P | 1.8 | 1.8 | 1.7 |
| Vitamins | Vit. A (μg RE) | 110 | 110 | 130 |
| (/100 kcal) | Vit. D (μg CE) | 1.6 | 1.6 | 1.7 |
| | Vit. E (mg) | 2.0 | 1.8 | 2.1 |
| | Vit. K1 (μg) | 11 | 11 | 8.0 |
| | Vit. C (mg) | 21 | 21 | 18.0 |
| | Vit. B1 (mg) | 0.10 | 0.10 | 0.15 |
| | Vit. B2 (mg) | 0.20 | 0.19 | 0.2 |
| | Niacin (mg) | 0.5 | 0.5 | 1.0 |
| | Vit. B6 (mg) | 0.05 | 0.05 | 0.10 |
| | Folic acid (μg) | 15 | 15 | 17.0 |
| | Pantothenic | 0.9 | 0.9 | 1.0 |
| | Acid (mg) | | | |
| | Vit. B12 (μg) | 0.2 | 0.2 | 0.3 |
| | Biotin (μg) | 2.2 | 2.2 | 3.0 |
| | Choline (mg) | 22 | 20 | 20.0 |
| | Inositol (mg) | 28 | 26 | 20.0 |
| | Taurine (mg) | 6.0 | 6.0 | 8.0 |
| | L-Carnitine (mg) | 1.7 | 1.7 | 1.6 |
| Trace | Fe(mg) | 0.85 | 0.85 | 1.0 |
| Elements | I (μg) | 20 | 20 | 24.0 |
| (/100 kcal) | Cu (mg) | 0.070 | 0.073 | 0.08 |
| | Zn (mg) | 1.0 | 1.0 | 1.2 |
| | Se (μg) | 3.0 | 3.3 | 2.9 |

The hydrolyzed whey protein of the PHw group and of the DISCH group are complex whey protein partially hydrolysed with an extend of hydrolysis of 10-15% amino-N/TN measured by TNBS method. They also have 80-90% NPN/TN.

The infant formula are in powder form. A volume of 600 mL of the product was orally ingested within 10 minutes.

Blood samples were taken from vein after an overnight fast and at different intervals during 2 hr post meal intake to measure the glucose response and the insulin response. The glucose/insulin responses were assessed at different time points before intake of the test meal drink (−10 & 0 minutes) and following start of test meal intake (15, 30, 45, 60, 90, 120, 150 and 180 minutes). Arterialized blood samples, taken at above mentioned points, were analyzed for plasma glucose and insulin
Glucose is analyzed by a Cobas C111 from Roche with the glucose oxidase technique (200 μL plasma), using Roche GLUC2 (04657527, Switzerland)
Insulin is analyzed by ELISA method with a highly specific and sensitive two-site enzyme-linked immunosorbent assay using IBL international insulin ELISA kit (RE53171, Germany).
C-peptide is analyzed by Elisa method with a two-site monoclonal-based using Millipore Human C-Peptide Elisa kit (EZHCP-20K, USA). C-peptide is a substance produced by the pancreas beta cells when proinsulin (inactive insulin) splits apart and forms one molecule of C-peptide and one molecule of insulin. Thus blood C-peptide concentration increase with increasing level of insulin production by pancreas.

The maximal concentration of glucose and insulin (Cmax) and the 2-hours incremental area under the glucose and insulin curves (2-h IAUC) were especially assessed, as known by the skilled person.

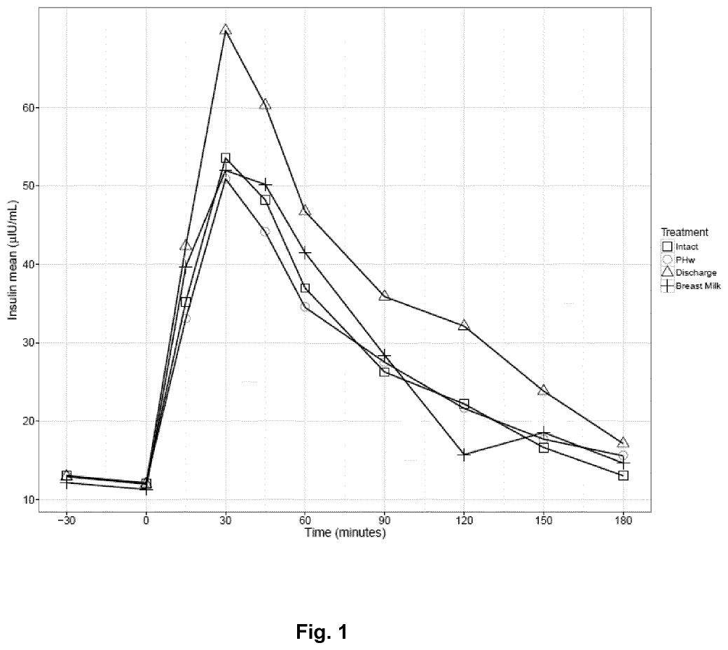

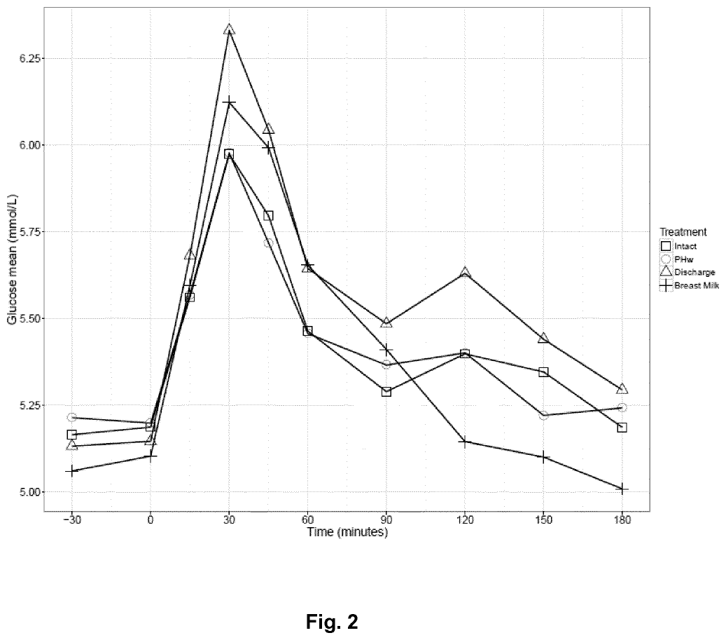

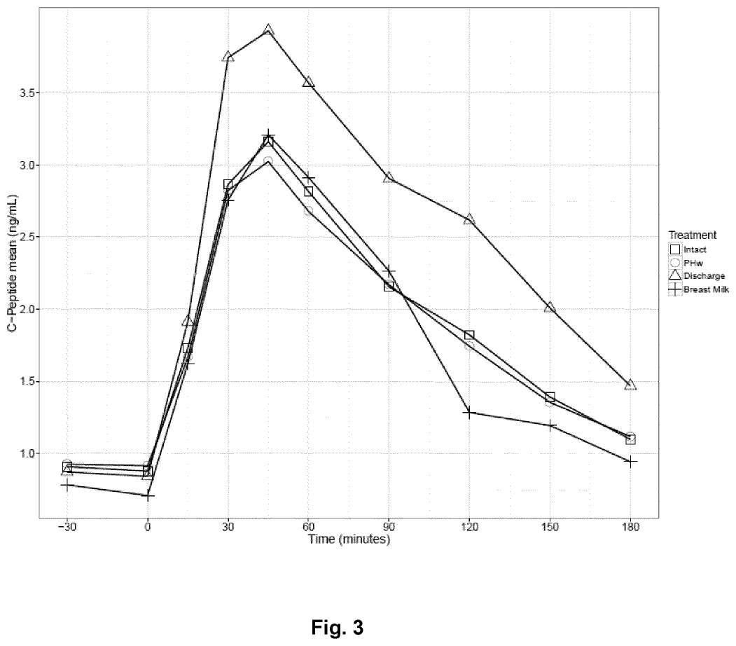

Results are illustrated in FIGS. 1, 2, 3 and 4.

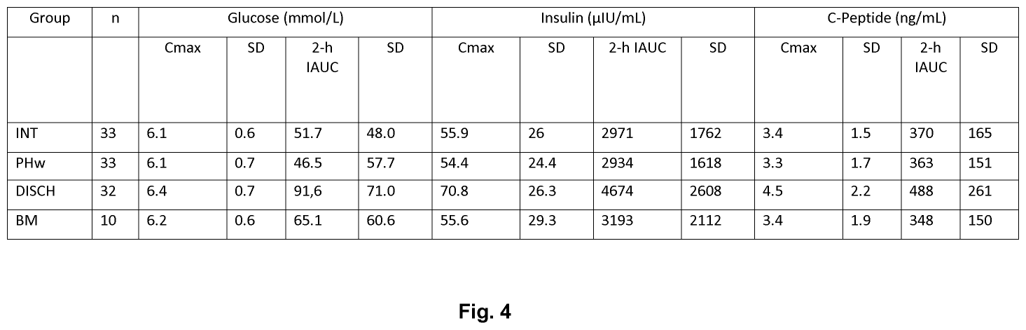

As it can be seen, the glucose, insulin and C-peptide responses of the INTACT group and the PHw group had surprisingly similar postprandial insulinaemia, glycaemia and C-peptide (Cmax and 2-h IAUC). The glucose, insulin and C-peptide responses of the PHw group were respectively not more than 10%, 2.6% and 2.9% different to the responses obtained for INTACT, for both C-max and 2-h IAUC comparisons (all were lower, except for C-max of glucose, see the table of FIG. 4). These responses were also surprisingly not statistically different from those to BM: the glucose, insulin and C-peptide responses of the PHw group were respectively not more than 29%, 8% and 4% different to the responses obtained for BM for both C-max and 2-h IAUC comparisons (all were lower, except for IAUC of C-peptide, see the table of FIG. 4).

The PHw formula, a nutritional composition comprising from 1.5 to 2.3 g/100 kcal of partially hydrolysed whey proteins appears to be very efficient to promote a glucose and an insulin responses that are similar to the glucose and insulin responses of individuals fed exclusively with human breast milk or also with intact proteins. There is not any significant increase of the glucose and insulin responses in comparison to the responses obtained for individuals fed exclusively with human breast milk or with intact proteins.

In addition, the DISCH formula (not part of the invention), which has a higher protein content, induces a higher (p<0.05) postprandial insulin and C-peptide responses relative to all other groups. The glucose response to DISCH formula was also higher than that of INTACT and PHw groups (P>0.05), even if not statistically different from BM. The 2-h IAUC of glucose, insulin and C-peptide responses of the PHw group were respectively 50%, 37% and 26% lower than the responses obtained for DISCH.

The PHw formula, a nutritional composition comprising from 1.5 to 2.3 g/100 kcal of partially hydrolysed whey proteins appears to be very efficient in promoting and/or inducing a glucose and an insulin responses that are lower than the glucose and insulin responses of individuals fed predominantly or exclusively with a nutritional composition comprising more than 2.3 g/100 kcal (e.g. 2.8 g/100 kcal) of partially hydrolysed whey proteins.

The PHw formula is also therefore thought to be particularly efficient for use in providing a healthy growth, for use especially in infants at risk of allergy in preventing a disorder linked to an increase in insulin secretion, for preventing obesity later in life, diabetes later in life and/or insulin resistance in individuals like infants or young children.

1. A method for use in infants or young children in promoting a glucose and/or an insulin response(s) that is/are similar to the glucose and/or insulin response(s) of infants or young children fed predominantly or exclusively with human breast milk and/or for use in preventing any significant increase of the glucose and/or insulin response(s) in comparison to the response(s) obtained for infants or young children fed predominantly or exclusively with human breast milk comprising administering to an infant or a child a nutritional composition comprising from 1.5 to 2.3 g/100 kcal of partially hydrolysed whey proteins.

2. A method for use in infants or young children in promoting a glucose and/or an insulin response(s) that is/are similar to the glucose and/or insulin response(s) of infants or young children fed predominantly or exclusively with intact proteins and/or for use in preventing any significant increase of the glucose and/or insulin response(s) in comparison to the response(s) obtained for infants or young children fed predominantly or exclusively with intact proteins comprising administering to an infant or a child a nutritional composition comprising from 1.5 to 2.3 g/100 kcal of partially hydrolysed whey proteins.

3. A method for use in promoting and/or inducing in infants or young children a glucose and/or an insulin response(s) that is/are lower than the glucose and/or insulin response(s) of infants or young children fed predominantly or exclusively with a nutritional composition comprising more than 2.3 g/100 kcal of partially hydrolysed whey proteins comprising administering a nutritional composition comprising from 1.5 to 2.3 g/100 kcal of partially hydrolysed whey proteins.

4. Method according to claim 1 wherein the composition comprising from 1.8 to 2.0 g/100 kcal of partially hydrolysed whey proteins.

5. Method according to claim 1, wherein the partially hydrolysed whey proteins have a % amino-N/TN value of 8-17%, such as 10-15%.

6. Method according to claim 1, wherein the partially hydrolysed whey proteins have a % NPN/TN value of 75-95%.

7-9. (canceled)

10. Method according to claim 1 wherein the nutritional composition also comprises caseins.

11. Method according to claim 1, wherein the nutritional composition comprises at least one probiotic in an amount of from 10³ to 10¹² cfu/g of said composition (dry weight).

12-16. (canceled)

17. Method according to claim 2 wherein the composition comprising from 1.8 to 2.0 g/100 kcal of partially hydrolysed whey proteins.

18. Method according to claim 2, wherein the partially hydrolysed whey proteins have a % amino-N/TN value of 8-17%, such as 10-15%.

19. Method according to claim 2, wherein the partially hydrolysed whey proteins have a % NPN/TN value of 75-95%.

20. Method according to claim 2 wherein the nutritional composition also comprises caseins.

21. Method according to claim 2, wherein the nutritional composition comprises at least one probiotic in an amount of from 10³ to 10¹² cfu/g of said composition (dry weight).

22. Method according to claim 3 wherein the composition comprising from 1.8 to 2.0 g/100 kcal of partially hydrolysed whey proteins.

23. Method according to claim 3, wherein the partially hydrolysed whey proteins have a % amino-N/TN value of 8-17%, such as 10-15%.

24. Method according to claim 3, wherein the partially hydrolysed whey proteins have a % NPN/TN value of 75-95%.

25. Method according to claim 3 wherein the nutritional composition also comprises caseins.

26. Method according to claim 3, wherein the nutritional composition comprises at least one probiotic in an amount of from 10³ to 10¹² cfu/g of said composition (dry weight).
